(19) United States
(12) Patent Application Publication
(10) Pub. No.: US 2008/0096271 A1
(43) Pub. Date: Apr. 24, 2008

(54) Polymeric self-assembly multiplayer membranes for ion screening: design, fabrication and application in sensors

(75) Inventors: Jicang Zhou, Shanghai (CN); Qing Li, Shanghai (CN)

Correspondence Address: DARBY & DARBY P.C., P.O. BOX 770, Church Street Station, New York, NY, 10008-0770, US

(73) Assignee: Honeywell International, Inc., Morristown, NJ (US)

(21) Appl. No.: 11/713,794

(22) Filed: Mar. 5, 2007

(30) Foreign Application Priority Data
Oct. 20, 2006 (CN) 2006101356818

(51) Int. Cl. C08J 5/20; B05D 1/36; C12M 1/34

(52) U.S. Cl. 435/287.1; 427/402; 521/27

(57) ABSTRACT

The embodiments of the invention are related to an extrinsically compensated multilayer membrane comprising at least a first layer comprising charged polymers and a second layer comprising neutral polymers, wherein the first layer is characteristic of extrinsic compensation and the charges on the polymer are balanced by mobile counter ions. The embodiments of the invention relate to: i) extrinsically compensated membrane for ion screening; ii) two types of polymers for fabricating the extrinsically compensated membrane; iii) driving force for self-assembly and approaches to create such a driving force between the two polymers, which involve the functionalization of the two types of polymers via functional moieties: donors and acceptors; iv) one or more approaches of synthesis of the two types of polymers; v) the fabrications of extrinsically compensated membrane with the two types of polymers; and vi) the fabrication of functional sensing membranes and the sensors thereof.

### RELATED APPLICATIONS

This application is a national stage application of Chinese Application No. 2006101356818, filed on Oct. 20, 2006. The disclosure of the prior application is considered part of and is incorporated by reference in the disclosure of this application.

### FIELD OF INVENTION

The embodiments of the invention relate to an extrinsically compensated multilayer membrane.

### BACKGROUND

Functional membranes have applications in various areas, among these separations and sensors are two types of important applications. In sensor applications, functional membranes are usually deposed on and bound to the surface of transducers such as ion-selective field effect transistor (ISFET), ion-selective electrode (ISE), optic fibers, micro cantilevers, surface acoustic wave (SAW), bulk acoustic wave (BAW), and quartz crystal microbalance (QCM), etc. The most important function of such membranes is their capability of sensing, specifically, they need to recognize analytes of interests and generate signals that can be detected by transducers. Typically such a membrane is fabricated via incorporation of functional materials into a matrix. As one example a sodium sensitive membrane can be made via incorporating sodium-ionophore into a plasticized polyvinylchloride (PVC) matrix; as another example a urea sensitive membrane can be fabricated via embedding an enzyme (urease) into a polyvinyl alcohol (PVA) matrix to name a few. In addition, ideal functional membranes deposed on the transducer surface should selectively limit or reduce the flux or pass of some undesirable components in a solution to reach the transducers, greatly enhancing the sensor performance (such as the sensitivity, selectivity, dynamic range and response time).

Recently, there has been a particular interest in developing biosensors for real time monitoring of human metabolic parameters like urea, creatinine, glucose, etc. Functional sensing membranes are extremely important in the fabrication of such biosensors. Typical fabrication of such membranes involves the encapsulation of functional materials into a polymer matrix, for example, an enzyme (urease) is encapsulated into a PVA matrix to make a urea sensitive membrane. Here, the major challenge associated with the functional membranes is the technical strategy to reduce flux of or screen certain ions, such as HCO₃—, Na+ and so on from the bulk solutions, which in some cases have undesired effects on the performance of the membranes and the sensors.

Various approaches have been explored for the fabrication of functional membrane for sensing applications. Recently, one witnessed the design and fabrication of multilayer functional membranes for sensing applications. The approach involves the encapsulation of functional materials (e.g., enzyme) into self-assembly multilayer membranes. The functional materials is to recognize analytes of interests and produce signals to be detected by transducers; while the multilayer membranes, besides stabilizing the encapsulated functional materials (e.g., enzyme), is to screen the undesired ions in bulk solutions. Typically, multiplayer membranes were based on layer-by-layer self-assembly approach which involves the consecutive self-assembly of two types of polyelectrolytes, i.e., polyanions and polycations. For example, if one starts with a substrate with positive charges in the fabrication process, polyanions were first adsorbed onto this surface to make the surface negatively charged. Thereafter, polycations were adsorbed onto the surface to make the surface positive charged again. Repeating this cycle again and again could produce a membrane with alternating polyanion/polycations layer (which means a self-assembly multilayer membrane).

The terms “intrinsic compensation” and “extrinsic compensation” characterize the layer-by-layer self-assembly membranes, particularly its ion-screening capability. If charges on one polymer are balanced (or neutralized) by opposite charges on another polymer, such as polycations balanced by polyanions or vice versa, it is called “intrinsically compensated.” On the other hand, if charges on one polymer are balanced by opposite charges of mobile ions (not by charges on polymers which can not move freely), such as polycations by free-moving anions in solution or polyanions by free-moving cations in solution, it is called “extrinsically compensated.” The membrane described in the previous paragraph is intrinsically compensated as the charges in the layer are neutralized by the counterpart polyelectrolyte (namely, positive charge of a polycation would be neutralized by negative charge of a polyanion). In all existing technologies for making ion-screening multilayer membranes, charges on the two polymers are all intrinsically compensated and no multilayer membranes based on extrinsic compensation have been reported.

The existing intrinsically compensated layer-by-layer self-assembly approach shown in FIG. 1 is commonly used because of its simplicity. While such multilayer membranes have been successfully used to screen some types of ions, such as screening [HCO₃⁻] from a common carbonate buffer with a high buffer capacity, its performance still needs to be improved to meet requirements of some specific applications, e.g., functional sensing membranes for urea, creatinine, and glucose, etc.

This invention solves the problem associated with the ineffective ions screening of the commonly used, intrinsically compensated multilayer functional membranes. The invention describes a novel type of ion-screening multilayer functional membrane with extrinsically compensated, charged polymeric layers that are bound together by neutral polymeric layers and with a high ion-screening capabilities, and the invention also provides methods to design and fabricate such membranes.

### BRIEF DESCRIPTION OF THE DRAWINGS

FIG. 1 shows the prior art of fabricating intrinsically compensated layer-by-layer self-assembly multilayer membrane.

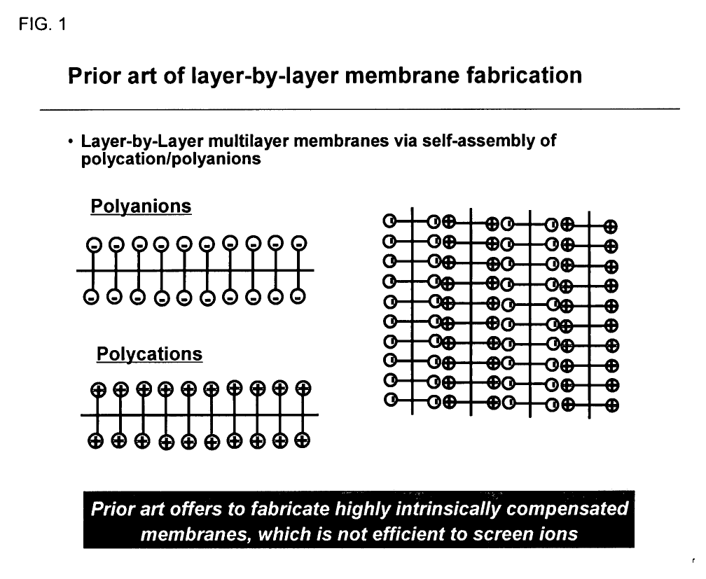

FIG. 2 shows the fabricating of an extrinsically compensated layer-by-layer self-assembly multilayer membrane comprising layers of charged polymer and neutral polymer with functional group donors and acceptors.

FIG. 3a a diagram of a possible configuration of a sensor device with functional sensing membranes immobilized onto the transducer surface; FIG. 3b is a diagram of the possible configurations of functional sensing membrane comprising sensing materials and an extrinsically compensated layer-by-layer multiplayer structure. Case I shows functional sensing membrane via the one-step approach, i.e., directly incorporating of functional materials into the layer-by-layer multilayer membrane during self-assembly. Case II shows functional sensing membrane via the two-step approach. i.e., first spin-coating a sensing layer containing functional materials and then fabricating the layer-by-layer multilayer membrane.

### SUMMARY OF THE INVENTION

The embodiments of the invention relate to the design and fabrication of a multilayer functional sensing membrane that is deposited on the transducer of a sensor. The membrane can selectively reduce the flux or pass of some undesirable ions in solutions (e.g., buffer solutions) to reach the transducer surface and therefore enhance the sensor's performance. More particularly, the invention pertains to a multilayer membrane with layer-by-layer structures in which two types of layers exist. That is, the first type of layers comprises charged (negative or positive) polymers and the second type of layer comprises neutral polymers. The two types of layers are ordered in an alternating way. This type of membrane is characteristic of extrinsic compensation and could be very useful in sensor fabrication.

The embodiments of this invention solve the problem of the ineffectiveness of the commonly used membranes, e.g., intrinsically compensated multilayer functional membranes, in screening some undesirable ions from a solution, e.g., buffer solutions. The invention describes a novel type of ion-screening multilayer membrane with extrinsically compensated, charged polymeric layers that are bound together by neutral (no charge) polymeric layers and with a high ion-screening capabilities, and the invention also provides methods to design and fabricate such membranes.

The embodiments of this invention pertains to approaches on the design and synthesis of polymers that can be used to fabricate extrinsically compensated functional multiplayer self-assembly membranes.

The embodiments of this invention are also associated with the fabrication of high performance sensors with sensing membranes containing extrinsically compensated multilayers. The embodiments of this invention describe two configurations for the sensing membranes. In the first preferred embodiment, the sensors are fabricated via spin-coating of a mixture of sensing materials (e.g., enzyme like urease) and polymer (e.g., PVA) onto a transducer's surface followed by a second coating of an extrinsically compensated layer-by-layer self-assembly multilayer membrane described above. In the second preferred embodiment, the sensors are fabricated via directly incorporating the sensing materials (e.g., enzyme) into the extrinsically compensated layer-by-layer self-assembly multilayer membrane during the self-assembly process.

As would be realized, this invention is capable of other and different embodiments, and its details are capable of modifications in various obvious respects, all without departing from this invention. Accordingly, the drawings and description are to be regarded as illustrative in nature and not as restrictive.

### DETAILED DESCRIPTION

As used in the specification and claims, the singular forms “a”, “an” and “the” include plural references unless the context clearly dictates otherwise.

The term “functional membrane” refers to is a membrane having a functional polymer and which would allow certain molecules or ions to pass through it by diffusion or facilitated diffusion. The term “functional polymer” refers to a polymer that bears a specified chemical group and has a specified physical, chemical, biological, pharmacological, or other uses that depend on the specified chemical group. The term “facilitated diffusion” refers to a diffusion process in which molecules diffuse across the membrane, with the assistance of transporting agent such as a molecule (e.g., protein).

A “macromolecule” or “polymer” comprises two or more monomers covalently joined. The monomers may be joined one at a time or in strings of multiple monomers, ordinarily known as “oligomers.” Thus, for example, one monomer and a string of five monomers may be joined to form a macromolecule or polymer of six monomers. Similarly, a string of fifty monomers may be joined with a string of hundred monomers to form a macromolecule or polymer of one hundred and fifty monomers. The term polymer as used herein includes, for example, both linear and cyclic polymers, heteropolymers in which a compound is covalently bound to a polymer, and a biopolymer.

The term “biopolymer” refers to a special class of polymers found in nature. Starch, proteins and peptides, DNA, and RNA are all examples of biopolymers, in which the monomer units, respectively, are sugars, amino acids, and nucleic acids. Biopolymers include a nucleotide, a polynucleotide, an oligonucleotide, a peptide, a protein, a ligand, a receptor, among others.

The term “polycationic” refers to a molecule with a plurality of positively charged groups. The term “polyanionic” refers to a molecule with a plurality of negatively charged groups.

The embodiments of the invention relate to a new charged polymeric multilayer membrane that is extrinsically compensated and have a much higher ion-screening capability than the commonly used membranes, e.g., intrinsically compensated layer-by-layer self-assembly membrane. The enhancement of ion-screening capability by an extrinsically compensated multilayer membrane has not been disclosed in the prior art.

Our results are based on the mathematical modeling of the multilayer systems. Our modeling includes three types of basic equations: (1) charge neutrality equations for each layer, (2) chemical potential balance equations for each chemical component and for each layer, and (3) balance equations for mixing, elastic and ionic free energies for each layer. The results of solving these equations indicate a significant increase in ion-screening capability by extrinsically compensated layer-by-layer membranes compared with intrinsically compensated ones. To bind together two or more layers of extrinsically compensated charged polymers, a neutral-charge polymeric layer is used.

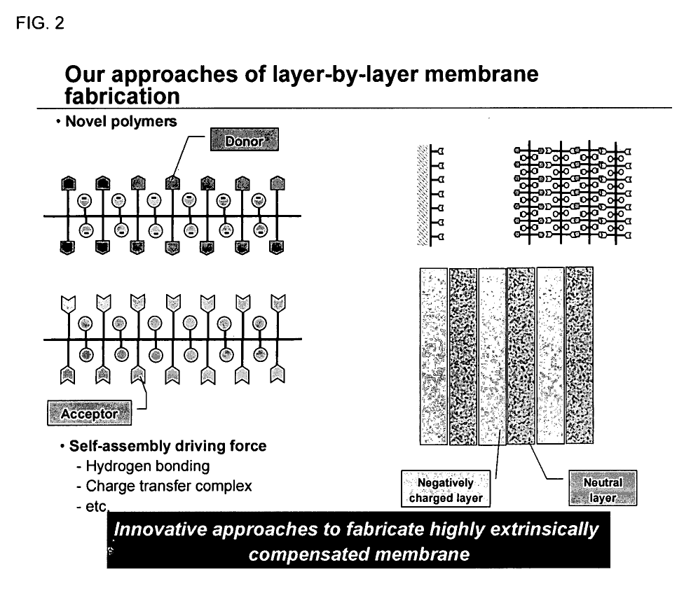

The embodiments of this invention relate to a novel extrinsically compensated layer-by-layer multilayer membrane which is exemplified in FIG. 2. A point of novelty of the embodiments of the invention is that this invention is the first to invent a new type of polymeric multilayer membranes that is substantially extrinsically compensated and has a much higher ion-screening capability than the commonly used membranes, e.g., intrinsically compensated multilayer membrane. The idea of using a neutral polymer layers to bind together two layers of extrinsically compensated polymers with the same charges (polyanions or polycations) is also novel.

The embodiments of the invention relate to the fabrication of a multiplayer polymer membrane with extrinsic compensation in the layers. The fabrication of the multilayer membrane could start with a polyelectrolyte (polycation or polyanion) and a neutral polymer, both possess functional moieties (groups) to facilitate the self-assembly process. For example, if one wants to fabricate a membrane with polycations and a neutral polymer, the process of fabrication could be as follows. Start with a substrate with proton-donating groups on the surface. Then adsorb a polycation with proton-accepting groups onto the surface and rinse the surface. Thereafter, adsorb a neutral polymer with proton-donating groups onto the polycations. Repeating the process would produce the so called “layer-by-layer self-assembly multilayer membranes”. Membrane thereof is extrinsically compensated as the charges of the polymers in the membrane are neutralized by mobile counter ions, instead of the counterpart polyelectrolytes.

The differences between intrinsically compensated and extrinsically compensates membranes are very clear. First, different starting materials are used in the fabrication of the two types of layer-by-layer self-assembly multilayer membranes. In the case of extrinsically compensated membranes, a polyelectrolyte (polycation or polyanion) and a neutral polymer are used, while in the case of intrinsically compensated membranes, a polycation and a polyanion are used. Secondly, the driving force of the self-assembly process is different. In the case of extrinsically compensated membranes, driving force may include hydrogen bonding, chemical bonding, and so on; while in the case of intrinsically compensated membrane, the driving force is the ionic interaction between the polyanion and polycation. Finally, the electrical neutralization of the charged polymers in the membrane is different as well. In the case of intrinsically compensations, charges on one polymer are balanced (or neutralized) by opposite charges on another polymer, such as polycations balanced by polyanions or vice versa; while in the case of extrinsic compensation, charges on one polymer are balanced by opposite charges of mobile counter ions (not by charges on polymers which can not move freely), such as polycations by mobile counter anions in solution or polyanions by mobile counter cations in solution.

An ion-screening layer contains preferably only one type of polyelectrolyte (charged polymers, either negative or positive) with some special functional moieties. The choice of the types of polyelectrolytes depends on the types of the ion to be screened. Specifically, a negatively charged polymer (polyanions) is preferred to screen anions like HCO₃—, HSO₃—, etc., and positively charged polymers (polycations) are preferred to screen cations like Na+, Ca₂+, etc. The special functional moieties are needed to facilitate the self-assembly of the charged polymers with a second type of polymers that are typically neutral polymers containing a second types of functional moieties. The neutral layers containing neutral polymers separate the charged layers, forming a multilayer structure and consequently increasing the ion-screening capability of the multilayer membranes.

In the embodiments of the invention, both charged polymers and neutral polymers have functional moieties or groups to facilitate the self-assembly and bind together these two types of polymers. Functional moieties include those groups that can form hydrogen bonding, chemical bonding, and so on. In one preferred embodiment, hydrogen bonding is the driving force for self-assembly. In such a case, functional moieties may include the following: —OH, —COOH, —NH₂, etc., one skilled in the art can list other functional groups capable of forming hydrogen bonding. In another preferred embodiment, chemical bonding is the driving force for self-assembly. In such a case, moieties may include —OH, —COOH, —NH₂, —CHO, etc., one skilled in the art can list other moieties capable of forming chemical bonding.

For simplicity, such functional moieties can be divided into two types, respectively termed as donating groups (“donor”) and accepting groups (“acceptor”). Either charged polymers can have donors and neutral polymers have acceptors, or on the contrary, charged polymers have acceptors and neutral polymers have donors. The bonding forces between donors and acceptors bind the charged polymeric layer with the neutral polymeric layer.

Two approaches can be used to incorporate functional groups, i.e., donor and acceptor, into the desired polyelectrolytes (polycations and polyanions) and neutral polymers. In one preferred embodiment, the functional groups are incorporated in the polymer via copolymerization of the functional monomers (monomers containing the desired functional moieties (groups), i.e., donors or acceptors) with the second monomer (cationic monomers, anionic monomers, or nonionic monomers). Various monomers containing donors or acceptors are known, for examples, 4-vinylpyridine, 4-vinylpyrrolidone, 4-vinylphenol, 2-hydroxyethyl(meth)acrylate, 4-vinylaniline, 4-vinylbenzaldehyde. One skilled in the art would be able to list more functional monomers. In another preferred embodiment of this invention, the functional moieties (group) may be incorporated into the desired polymer via modification of available polymers with desired donors or acceptors. One skilled in the art would be able to design the chemical reactions and reagents for such purpose.

To make the embodiments of the multilayer membranes, for example, as shown in FIG. 2, two types of polymers could be designed and synthesized. The first type of polymers could be either negatively or positively charged and has donors or acceptors to facilitate the self-assembly with the second type of polymers. The second type of polymers is typically neutral polymers containing donors and acceptors to facilitate self-assembly with the first type of polymers.

The embodiments of this invention relate to the synthesis of polycations with functional moieties (donors or acceptors) for the fabrication of extrinsically compensated layer-by-layer self-assembly multilayer membranes. In one preferred ernbodiment, the polymers can be made via copolymerization of cationic monomers which offer the polymer the cationic characteristics and functional monomers which offer the polymer the capability of forming hydrogen bonding, chemical bonding, etc with the counterpart neutral polymer. Various cationic monomer are available to fabricate such polymers, for examples, diallyldimethylammonium chloride, N,N-dimethylaminoethyl acrylate methyl chloride quaternary, acryloxyethyidimethylbenzyl ammonium chloride. One skilled in the art would be able to list more cationic monomers. In another preferred embodiment, the polycations may be made via modifying available polycations with functional groups via chemical reactions. Available polycations include natural polycations or synthetic polycations, e.g., polyethyleneamine (PEI). One skilled in the art would be able list some polycations capable of further modification to make the desired polymers. One skilled in the art would be able to design approaches to modify such polycations with functional moieties like donors or acceptors.

The embodiments of this invention relates to the synthesis of polyanions with functional moieties for the fabrication of extrinsically compensated layer-by-layer self-assembly multilayer membranes. In one preferred embodiment, such polymers can be made via copolymerization of anionic monomers which offer the polymer the anionic characteristics and functional monomers which offer the polymer the capability of forming hydrogen bonding, chemical bonding with the counterpart neutral polymer. Various anionic monomers are available to fabricate such polymers, for examples, beta-carboxyethyl acrylate, sodium 1-allyloxy-2-hydroxy propane sulphonate, ammonium allylpolyethoxy (10) sulphate, sodium acrylate, sodium methacrylate. One skilled in the art would be able to list more of such monomers. In another preferred embodiment, the polycations may be made via modifying available polyanions with functional groups via chemical reactions. Available polyanions include natural polyanions or synthetic polyanions, e.g., polyacrylic acid. One skilled in the art would be able name some polyanions capable of further modification to make the desired polymers. One skilled in the art would be able to design approaches to modify such polyanions with functional moieties like donors or acceptors.

The embodiments of this invention also relate to the fabrication of neutral polymers with functional moieties for the fabrication of extrinsically compensated layer-by-layer self-assembly multilayer membranes. In one preferred embodiment, the neutral polymers would be made via copolymerization of nonionic monomers which offer the polymer the nonionic characteristics and functional monomers which offer the polymer the capability of forming hydrogen bonding, chemical bonding, etc with the counterpart polycations or polyanions. Various anionic monomers are available to fabricate such polymers, for examples, 2-hydroxyethyl(meth)acrylate, dimethylaminoethyl(meth)acrylate, acrylamide, N-isopropyl acrylamide. One skilled in the art would be able to list more nonionic monomers. In another preferred embodiment, the neutral polymer may be made via modifying available neutral polymers with functional groups via chemical reactions. Available neutral polymers include some natural or synthetic polymers, e.g., PVA, cellulose, etc. One skilled in the art would be able to name some neutral polymers capable of further modification to make the desired polymers. One skilled in the art would also be able to design approaches to modify such neutral polymers with functional moieties like donors or acceptors.

With the two types of polymers having been designed and synthesized, the extrinsically compensated multilayer functional membrane can be fabricated. The embodiments of this invention pertain to the fabrication of extrinsically compensated multilayer membranes via layer-by-layer self-assembly. In one preferred embodiment, the driving force for the self-assembly involves the hydrogen bonding. For example, if one wants to fabricate a membrane with a polycation and a neutral polymer containing proton-accepting groups and proton-donating groups, respectively, the process of fabrication would be as follows. One could start with a substrate with proton-donating groups on the surface, then a polycation with proton-accepting groups is adsorbed onto the surface via hydrogen bonding, this is then rinsed to remove the non hydrogen bonded polymers. Thereafter, a neutral polymer with proton-donating groups was adsorbed onto the polycation layers via hydrogen bonding and rinsed again. Repeating the process could produce a multilayer membrane with alternating polycation/neutral polymer layers. One skilled in the art would understand that other procedures can also be used to fabricate layer-by-layer self-assembly multilayer membrane with hydrogen bonding as the driving force. In another preferred embodiment, the driving force for the self-assembly involves chemical bond formation between the two polymers. For example, if one wants to fabricate a membrane with a polycation and a neutral polymer containing donors (e.g., —NH₂) and acceptors (e.g., —CHO), respectively, the process of fabrication can be as follows. For example, one could start with a substrate with —CHO groups on the surface. Then a polycation with —NH₂ groups could be adsorbed onto the surface via the formation of a Schiff base bonding between the two functional groups, followed by rinsing to remove the non chemically bonded polycations. Thereafter, a neutral polymer with —CHO groups could be adsorbed onto the polycation layers via chemical bonding formation and rinsed again to remove the non chemically bonded neutral polymers. Repeating the process could produce a multilayer membrane with alternating polycation/neutral polymer layers. One skilled in the art would understand that other procedures can also be used to fabricate layer-by-layer self-assembly multilayer membranes using chemical bonding as the driving force.

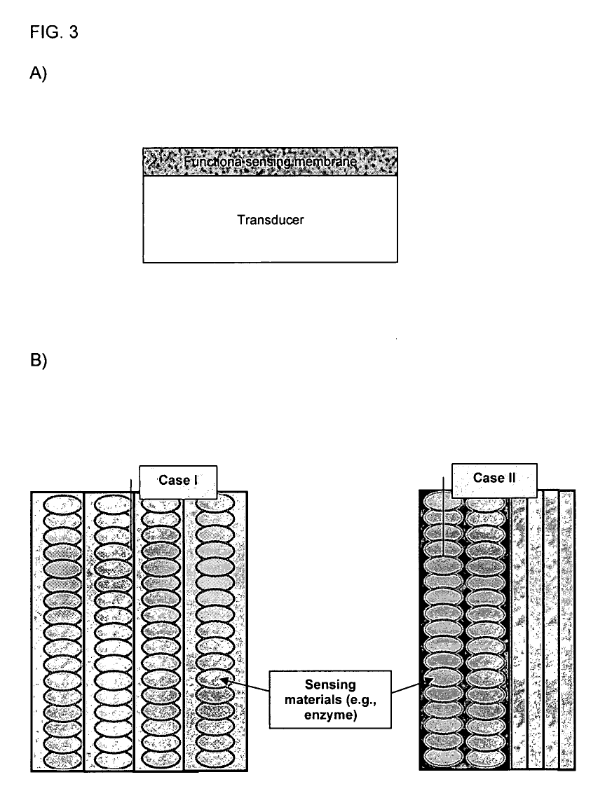

The embodiments of the extrinsically compensated multilayer membranes of the invention can be very useful in sensors fabrications. The membranes have high ion-screening capabilities and therefore can enhance the performance of the sensor. Moreover, this invention also describes the methods to make such functional sensing membranes. FIG. 3a shows a diagram of a possible configuration of a sensor device with functional sensing membranes immobilized onto the transducer surface; FIG. 3b shows a diagram of the possible configurations of functional sensing membrane comprising sensing materials and an extrinsically compensated layer-by-layer multiplayer structure.

The preferred embodiment of the invention is a sensor that has the above membranes to screen or limit flux of some undesired ions in a solution from reaching the sensing layer or transducer surface. The sensors can be fabricated via modifying the transducer surface with a functional sensing membrane. In one preferred embodiment, the transducer is an ISFET device. In another preferred embodiment, the transducer is an ISE. In another preferred embodiment, the transducer is an optic fiber. One skilled in the art would understand that other transducers may be used for sensors fabrications as well.

The embodiments of this invention pertain to the fabrication of sensors with a functional sensing membrane containing an extrinsically compensated multilayer membrane to screen the undesired ions in bulk solutions like buffers. In the preferred embodiment of this invention, the functional sensing membranes contain functional materials that can recognize the analytes of interest and produce signal that can be detected by the transducers.

Various functional materials can be used in the functional sensing membrane fabrication. In one preferred embodiment, the functional materials can be enzymes which selectively bind and decompose the analytes of interests and produce end-products that can be detected by certain transducers. For examples, urease, creatinease, glucose oxidase can be used to make functional sensors of urea, creatinine and glucose. One skilled in the art would understand that more anayltes can be recognized and decomposed by its corresponding enzymes and therefore the enzymes can be used as functional materials for sensing. It is well known that certain enzymes can decompose the compounds like urea, creatinine, and glucose, etc., and produce end-products like CO2, NH3, etc. Such end-products can be easily hydrolyzed, resulting in pH variation of the environment and meanwhile forming ions like ammonium ion, etc. The changes can be easily detected by transducers like ISFET, ISE, and so on. In another preferred embodiment, the functional materials can be a combination of enzymes with a suitable ionophore (also named as “ion carrier”). For example, the urease and ammonium ionophore, nonactin, can be used as functional materials to fabricate a urea sensor. Here the urease decomposes urea to form CO2 and NH3, which are subsequently hydrolyzed to ammonium ions. The ammonium ionophore, nonactin, can then bind the ammonium ion which can be detected by a transducer. In another preferred embodiment, the functional materials can be a combination of enzyme and a pH sensitive chromophore, e.g., 2′,7′-bis-(2-carboxyethyl)-5-(and-6)-carboxyfluorescein (BCECF acid) or 2′,7′-bis-(2;-carboxyethyl)-5-(and-6)-carboxyfluorescein, acetoxymethyl ester (BCECF, AM). For example, in the above case, a pH sensitive chromophore in stead of an ammonium ionophore can be used to fabricate the urea sensing membrane. In all the sensing processes described above, various ions were produced and detected by a certain type of transducer. Therefore it is necessary to screen the potential interfering ions in bulk solutions from transport through the sensing membrane to reach the transducer surface.

The embodiments of this invention relate to sensors with functional sensing membrane containing an extrinsically compensated multilayer membrane to screen undesired ions from the bulk solutions like buffers. A sensor thereof could have a much better performance (higher sensitivity, selectivity, wider dynamic range, and shorter response time) than those fabricated with other techniques.

There are two configurations for the functional sensing membranes. In the first preferred embodiment, the functional sensing membrane was made via a two-step approach. Specifically, a sensing layer was first deposited onto the transducer surface and subsequently a multilayer ion-screening or flux-limiting membrane was deposited over the sensing layer via the layer-by-layer self-assembly approach. In one preferred embodiment, the sensing layer contains functional materials, e.g.,.enzymes, embedded in the matrix like polymer. In another preferred embodiment the sensing layer contains a combination of enzymes and ionophores. In another preferred embodiment, the sensing layer contains enzymes and pH sensitive chromophores. In the second embodiment for the sensing multilayer membrane configuration, the sensing functional membrane was fabricated via directly incorporating of the sensing materials, e.g., enzyme into the layer-by-layer self-assembly multilayer membranes.

The embodiments of the inventions pertain to the fabrications of functional sensing membrane over transducers surface via a two-steps approach. First a sensing layer was coated onto the transducer surface via deposition of a mixture of functional materials (e.g., enzyme) and a polymer, e.g., PVA. In one preferred embodiment, this sensing layer was then stabilized via crosslinking of the enzyme and the polymer. Secondly, the ion-screening extrinsically compensated multilayer was fabricated onto the sensing layer using the layer-by-layer self-assembly approach described above.

The embodiments of this invention also pertain to the fabrication of functional sensing membranes over transducers surface via a one step approach. In one preferred embodiment, the functional materials was encapsulated the multilayer membranes. One skilled in the art understands well how to fabricate such a functional sensing membrane.

The extrinsically compensated multilayer membrane could have an ion screening value as high as 100%, preferably greater than 50%, more preferably greater than 75%, and most preferably greater than 95%. This ion screening factor would be determined by various membrane factors like the number of the layers and the swellability of the membrane. Our modeling results indicate that the ion screening capability increases with the number of layers but decreases with the swellability of the membranes. One skilled in the art would understand how to control the swellability of the membrane.

The application of extrinsically compensated polymeric multilayer membranes to fabricate functional sensing membrane over transducer surface is an innovative approach for ion screening. It has huge potential in the sensor development such as biosensors, medical sensors, and chemical sensors, etc. This brings a technical innovation in the fabrication technology of bio/chemical sensors with functional membrane as the sensing components.

The idea of using an extrinsically compensated polymeric multilayer membrane to screen ions may also have potential applications in other areas. For example, such membranes may have potential application in desalination, pharmaceutical industry, and so on. This means that this invention is also capable of other and different embodiments, and its details are capable of modifications in various obvious respects, all without departing from this invention.

1. An extrinsically compensated multilayer membrane comprising at least a first layer comprising charged polymers and a second layer comprising neutral polymers, wherein the first layer is characteristic of extrinsic compensation, wherein charges on the charged polymers are balanced by mobile counter ions.

2. The extrinsically compensated multilayer membrane of claim 1, wherein the mobile counter ions of the first layer are anions and in the membrane the mobile counter ions are balanced by cations on the charged polymers.

3. The extrinsically compensated multilayer membrane of claim 1, wherein the mobile counter ions of the first layer are cations and in the membrane the mobile counter ions are balanced by anions on the charged polymers.

4. The extrinsically compensated multilayer membrane of claim 1, wherein the second layer comprises a neutral polymer to bind the extrinsically compensated multilayer together and form an alternating layer-by-layer structure.

5. The extrinsically compensated multilayer membrane of claim 1, wherein the charged polymers and the neutral polymers comprise functional groups donors and acceptors which can form hydrogen bonding and/or chemical bonding to bind the first and second layers together.

6. The extrinsically compensated multilayer membrane of claim 5, wherein the first layer comprises a negatively charged polymer with functional groups donors or acceptors.

7. The extrinsically compensated multilayer membrane of claim 5, wherein the first layer comprises a positively charged polymer with functional groups donors or acceptors.

8. The extrinsically compensated multilayer membrane of claim 6, wherein the negatively charged polymers are synthesized via copolymerization of anionic monomers with functional monomers comprising functional groups donors or acceptors.

9. The extrinsically compensated multilayer membrane of claim 6, wherein the negatively charged polymers are synthesized via modification of available polyanions with functional groups donors or acceptors.

10. The extrinsically compensated multilayer membrane of claim 7, wherein the positively charged polymers are synthesized via copolymerization of cationic monomers with functional monomers with functional groups donors or acceptors.

11. The extrinsically compensated multilayer membrane of claim 7, wherein the positively charged polymers are synthesized via modification of available polycations with functional groups donors or acceptors.

12. The extrinsically compensated multilayer membrane of claim 5, wherein the neutral polymers are synthesized via copolymerization of neutral monomers with functional monomers with functional groups donors or acceptors.

13. The extrinsically compensated multilayer membrane of claim 5, wherein the neutral polymers are synthesized via modification of available neutral polymers with functional groups donors or acceptors.

14. A method of fabricating the extrinsically compensated multilayer membrane of claim 1 via layer-by-layer self-assembly, wherein the self-assembly driving force involves hydrogen bonding and/or chemical bonding between a charged polymer layer and a neutral polymer layer.

15. The method of claim 14, wherein the self-assembly driving force involves the hydrogen bonding between a charged polymer layer and a neutral polymer layer and the fabrication comprises obtaining a substrate with proton-donating groups on a surface, adsorbing a charged polymer with proton-accepting groups onto the surface via hydrogen bonding, and then adsorbing a neutral polymer with proton-donating groups onto the charged polymer layer via hydrogen bonding, and repeating the process to make the layer-by-layer self-assembly multilayer membrane

16. The method of claim 14, wherein the self-assembly driving force involves the chemical bonding between a charged polymer layer and a neutral polymer layer and the fabrication comprises obtaining a substrate with a first functional group on a surface, adsorbing a charged polymer with a second functional group onto the surface via chemical bonding between the two types of functional groups, and then adsorbing a neutral polymer containing a first type of functional group onto the charged polymer layer via chemical bonding, and repeating the process to make the layer-by-layer self-assembly multilayer membrane.

17. A sensor device with a functional sensing membrane immobilized onto a transducer surface, wherein the sensing membrane comprises sensing materials and an extrinsically compensated multilayer membrane.

18. The sensor device of claim 17, comprising a transducers selected from the group consisting of ion-selective field effect transistor (ISFET), ion-selective electrode (ISE), optic fiber, cantilever, surface acoustic wave (SAW), bulk acoustic wave (BAW), and quartz crystal microbalance (QCM).

19. The functional sensing membrane immobilized onto a surface of a transducer, wherein the functional sensing membrane comprises a sensing material and an extrinsically compensated multilayer membrane, wherein the sensing material is adapted to recognize analytes and produce a signal to be detected by the transducer.

20. The functional sensing membrane of claim 19, wherein the extrinsically compensated multilayer membrane is adapted to screen undesired ions in solution.

21. The functional sensing membrane of claim 19, wherein functional sensing membrane is deposed on and bound to the surface of the transducer.

22. The method of making the functional sensing membrane of claim 19, comprising a one-step approach to fabricate the functional sensing membrane onto the surface of the transducer, wherein the functional material is encapsulated into multilayer membranes during fabrication of an extrinsically compensated layer-by-layer multilayer membrane via self-assembly.

23. The method of making the sensor device of claim 19, comprising a two-step approach to fabricate the functional sensing membrane onto the surface of the transducer, wherein a sensing layer is coated onto the surface of the transducer via deposition of a mixture of functional materials and a polymer and the extrinsically compensated multilayer membrane is fabricated onto a layer of the sensing material using a layer-by-layer self-assembly approach.
